(19) United States
(12) Patent Application Publication
(10) Pub. No.: US 2008/0319217 A1
(43) Pub. Date: Dec. 25, 2008

(54) Carbonic Acid Derivatives, Method for the Manufacture Thereof and the Use Thereof

(75) Inventors: Stefan Ingrisch, Truchtlaching (DE); Gerhard Albrecht, Tacherting (DE); Stefan Thaler, Trostberg (DE); Mathias Bauer, Trostberg (DE)

Correspondence Address: MORGAN, LEWIS & BOCKIUS LLP, 1701 MARKET STREET, PHILADELPHIA, PA, 19103-2921, US

(73) Assignee: Construction Research & Technology GMBH, Trostberg (DE)

(21) Appl. No.: 12/091,882

(22) PCT Filed: Oct. 25, 2006
(86) PCT No.: PCT/DE2006/001885

§ 371 Date: Apr. 28, 2008

(30) Foreign Application Priority Data
Oct. 27, 2005 (DE) 10 2005 051 375.1

(51) Int. Cl. C07C 69/96

(52) U.S. Cl. 558/260

(57) ABSTRACT

The invention relates to carboxylic acid derivatives which are obtained by reacting an unsaturated dicarboxylic acid anhydride (A) with a hydrophobic reactive component (B) that contains at least one group reacting to carboxylic acid anhydrides and has an average molecular weight of 200 to 50,000 Dalton. The inventive carboxylic acid derivatives are excellent as a substance for preventing efflorescences on surfaces of hardened cementitious construction materials and/or hydrophobizing the corresponding cementitious systems. Furthermore, said cementitious products absorb considerably smaller amounts of water as a result of the inventive additives such that frost damage and premature rusting of the reinforcing steel can be significantly reduced.

The present invention refers to carbonic acid derivatives, methods for the manufacture thereof and the use thereof as additives for cementitious construction materials (such as concrete or mortar), which are particularly used for water repellent finishing of substances and/or for suppressing efflorescence on surfaces of hardened cementitious construction materials.

A known problem in cement-based construction materials is the occurrence of so-called efflorescence, wherein a distinction is made between primary and secondary efflorescence. The first mentioned efflorescence is produced during hardening e.g. of concrete, wherein the capillaries of the green concrete are filled by an aqueous solution of the water-soluble substances of the cement, substantially calcium hydroxide. During hardening the calcium hydroxide reacts on the concrete surface with the carbon dioxide of the air under formation of hardly soluble calcium carbonate. Caused by the precipitation of calcium carbonate, the calcium hydroxide concentration at the capillary opening is lower than in the interior of the capillaries. Thus, new calcium hydroxide permanently reaches, caused by diffusion, from the deeper layers of the concrete to the capillary opening and in turn reacts with CO₂ to form calcium carbonate. The respective process only comes to a standstill if the capillary openings are closed by calcium carbonate. Primary efflorescences occur to a very strong extent if a film of condensation water is located on the concrete surface, since then the entire calcium hydroxide can distribute over the entire surface of the concrete and coat this surface after reaction with carbon dioxide with water-insoluble calcium carbonate.

Moreover, staining can also occur in the case of outdoor exposure of fully hardened concrete, which is generally designated as secondary efflorescence. The secondary efflorescences usually take 1 to 2 years, wherein a cause for this is considered to be the slow formation of water-soluble calcium hydrogen carbonate from calcium carbonate.

Since the optical appearance of such components afflicted with efflorescences is very much affected, particularly in the case of colored concrete products, many tests were performed to prevent or suppress this efflorescence by various measures.

According to the prior art, two fundamental options were proposed, which, however, did not lead to satisfactory results. On the one hand the surfaces of hardened cementitious or concrete products are provided with special coatings, wherein most of all different silicate and acrylate coatings were suggested. The disadvantage of this method is, however, the fact that these later coatings are relatively laborious and inefficient.

Due to this reason it was attempted to add suitable additives to the construction materials before their hardening, which shall prevent or suppress efflorescences.

It is known from DE 32 29 564 A1 to additionally use chalk e.g. in the form of an aqueous chalk slurry when manufacturing dyed concrete bricks. This shall shift the formation gradient of calcium carbonate at the surface in that excessive calcium carbonate is offered already at the beginning of the solidification process.

Finally, it is suggested according to EP 92 242 A1 to add surface-active polymers to the concrete to prevent efflorescences. These surface-active polymers shall irreversibly lose their surface activity during hardening of the concrete and shall thereby be converted to water-insoluble products.

In practice such hydrophobizing agents have not become accepted for unhardened construction materials, since they do not have a reliable effect under different weather conditions.

Furthermore, DE 199 05 488 A1 discloses a powdery polymer composition on the basis of polyethercarboxylates, which contains a water-soluble polymer and a fine-particulate mineral carrier material.

DE 198 08 314 A1 discloses the use of graft polymers as a flux material for aluminate-cement-containing binder suspensions.

DE 197 97 970 A1 discloses polycarbonic acid functional polyorganosiloxanes and the use thereof for treating tanned leather.

DE 43 26 772 A1 discloses reaction products of olefinic unsaturated carbonic acids and polyetheroles for use as demulsifiers for crude oil emulsions.

DE 41 16 111 A1 discloses nitrogen-containing surface active agents (surfactants, tensides), which are suitable for producing free-flowing solid dispersions, particularly colorant preparations for offset printing.

DE 31 26 213 discloses bisester of alkenyl succinic acids and ethylene oxide-propylene oxide block polymers and their use as demulsifiers for oil/water emulsions, as an anticorrosive and as dispergators for colorants.

DE 695 30 032 T2 discloses a method for demulsifying water-in-oil emulsions by using polyalkylene glycol derivatives.

GB 768 790 A discloses organosiloxanes and the use thereof as lubricants, corrosion inhibitors and emulsifiers.

EP 0 281 838 A2 discloses sulfosuccinamide acids of polyoxypropylene diamines and their use as emulsifiers.

EP 0 291 073 B1 discloses a cementitious additive, which comprises a copolymer of a polyoxyakylene derivative and maelic acid anhydride, a hydrolyzed product of the copolymer or a salt of the hydrolyzed product. Pol. Bull., 1994, Vol. 32, pages 173 to 179 discloses polydimethylsiloxanes, which are obtained by conversion of cyclic anhydrides and polydimethylsiloxanes with terminal hydroxypropyl or aminoalkyl groups.

The Chinese Journal of Synthetic Chemistry, 2005, vol. 13, no. 2, pg. 190 to 192 discloses the synthesis and emulsification of modified Tween-20.

It was the object of the present invention to provide an agent for preventing efflorescences on surfaces of hardened cementitious construction materials and/or for water repellent finishing, which does not reveal the cited disadvantages of the prior art but which efficiently and reliably prevents efflorescence of cementitious construction materials. This object was solved according to the invention by the use of a carbonic acid derivative according to claim 1, particularly by a carbonic acid derivative according to claim 13. A method of producing the carbonic acid derivative according to claim 13 is defined in claim 2. Further advantageous embodiments of the invention are defined in the dependent claims.

It was surprisingly shown that these carbonic acid derivatives are extremely well suitable as a means for preventing efflorescence and/or water repellent hydrophobizing of cementitious construction materials. Moreover, the cementitious products absorb considerably less water by the additives according to the invention, whereby damage due to freezing and a premature rusting of the reinforcing steel can be reduced.

The carbonic acid derivatives according to the invention can be obtained by reaction of an unsaturated dicarbonic acid anhydride (A) with a hydrophobic reactive component (B), which has a group that is reactive at least with respect to carbonic acid anhydrides and which has a mean molecular weight of 200 to 50,000 Dalton.

Maleic acid anhydride, succinic acid anhydride, itacon acid anhydride, phthalic anhydride, dimethyl maleic acid anhydride, methyl succinic acid anhydride as well as 2,2-dimethyl succinic acid anhydride are preferably used as unsaturated dicarbonic acid anhydrides.

Six compound classes selected from the group (B)(i), (B)(ii), (B)(iii), (B)(iv), (B)(v) and (B)(vi) can be used as hydrophobic reactive compound (B).

The reactive component (B)(i) consists of a polydimethylsiloxane of the general formula (I)

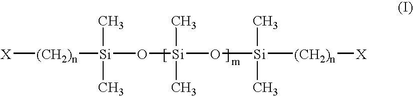

wherein

### X=OH, NH₂, SH, NHR¹,

### R¹=H, CH₃, C₂H₅,

m=1 to 50, preferably 10 to 30, and
n=1 to 6

As a reactive component (B)(ii) use can be made of a polypropylene glycol of the general formula (IIa)

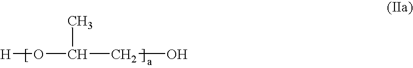

and/or a polypropylene glycol on the basis of a glycerol ether of the general formula (IIb)

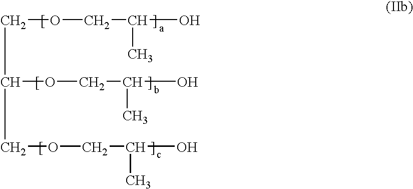

and/or a polypropylene glycol on the basis of a pentaerythritol ether of the general formula (IIc)

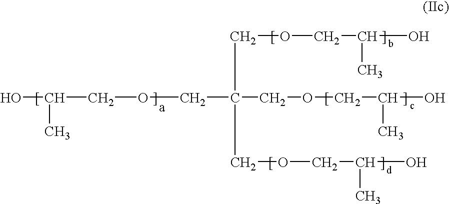

wherein
a, b, c and d means independent from one another 1 to 150.

The reactive component (B)(iii) either represents a polyoxyalkyleneamine of the formula (IIIa)

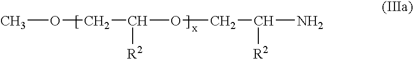

which are commercially available as Jeffamine® T-403, T-5000 as well as XTJ-509 or a polyoxyalkyleneamine of the general formula (IIIb)

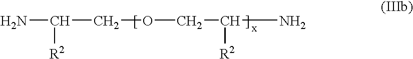

which are on the market as Jeffamine® D-230, D-400, D-2000 as well as D-4000 or a polyoxyalkylenetriamine of the general formula (IIIc)

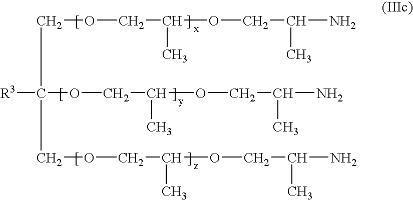

which are commercially available as Jeffamine® T-403, T-5000 as well as XTJ-509. In the general formulae (IIIa) and (IIIb)

### R²=H, CH₃,

### R³=H, CH₃, C₂H₅, and

x, y and z independent of one another 1 to 100.

As a reactive component (B)(iv) is used a polyaklylene glycol on the basis of alkylenediamines of the general formula (IV)

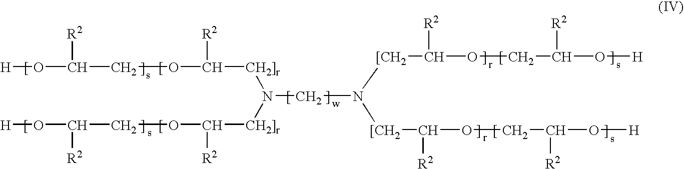

wherein
w=2 to 12 as well as
r and s represent independent of one another 1 to 150 and
R² has the above-mentioned meaning.

1,6-hexamethylenediamine, 1,8-octamethylenediamine, 1,10-diaminodecan as well as 1,12-diaminododecan are preferably used as alkylenediamines.

The reactive component (B)(v) consists of a triglyceride, which is composed of at least one hydroxy fatty acid: The preferred fatty acids that are used are ricinoleic acid, cerebronic acid, nemotin acid or 12-hydroxy stearic acid. The hydroxy fatty acid can possibly be etherified by 1 to 100 mol of an ethylene oxide derivative.

Finally, the reactive component (B)(vi) can be an epoxy derivative, which was produced by reacting a di, tri or tetraglycidyl component (C)(i) with a optionally unsaturated reactive component (C)(ii), consisting of a C₃-C₂₈-fatty acid, a C₈-C₂₈-alcohol or a C₈-C₂₈-secondary amine.

Glycidyl compounds (C)(i) are used as being especially advantageous, which are selected from the group of cyclohexane dimethanol diglycidylether, glycerine triglycidylether, neopentylglycol diglycidylether, pentaerythritol tetraglycidylether, 1,6-hexanediol diglycidylether, polypropylene glycol-diglycidylether, polyethyleneglycol diglycidylether, trimethylolpropane triglycidylether, bisphenol A diglycidylether, bisphenol F diglycidylether, 4,4′-methylenebis (N,N-diglycidylaniline), tetraphenylolethane glycidylether, N,N-diglycidylaniline, diethyleneglycol diglycidylether, 1,4-butanediol diglycidylether or mixtures thereof.

The reactive component (C)(ii) is consists of a C₈-C₂₈-fatty acid, a C₈-C₂₈-alcohol or a C₈-C₂₈-secondary amine, wherein the reactive component may have saturated or unsaturated residues.

Among the group of fatty acids, tall oil fatty acid, stearic acid, palmitic acid, sunflower oil fatty acid, coconut oil fatty acid (C₈-C₁₈), coconut oil fatty acid (C₁₂-C₁₈), soy bean oil fatty acid, linseed oil fatty acid, dodecane acid, oleic acid, linoleic acid, palm kernel oil fatty acid, palm oil fatty acid, linolenic acid and/or arachidonic acid must be regarded as being preferred. Among the C₈-C₂₈-alcohols, most of all 1-eicosanol, 1-octadecanol, 1-hexadecanol, 1-tetradecanol, 1-dodecanol, 1-decanol as well as 1-octanol have proven of value. In the secondary amines with C₈-C₂₈ C atoms, particularly the alkylamines of the group 2-ethylhexylamine, dipentylamine, dihexylamine, dioctylamine, bis(2-ethylhexyl)amine, N-methyloctadecylamine as well as didecylamine are used.

The reactive component (B)(vi) and the manufacture thereof are known according to the prior art (cf. e.g. the German patent application DE 10 2005 022 852 A).

The molar ratios in view of the unsaturated dicarbonic acid anhydride (A) and the hydrophobic reactive component (B) can be varied to a large extent. However, it has been proven to be especially advantageous that the molar ratio of the unsaturated dicarbonic acid hydride (A) to the reactive component (B) is 0.1 to 1.0 mol (A) per mol reactive group of component (B).

The manufacture of the carbonic acid derivative according to the invention is relatively unproblematic. According to a preferred embodiment, the unsaturated dicarbonic acid anhydride with the reactive component (B) is reacted without use of a solvent in the temperature range of 20 to 150° C. possibly in the presence of a catalyst. The catalyst that is used is preferably an alkaline or alkaline earth salt of an organic acid, such as sodium acetate or potassium acetate.

The carbonic acid derivatives used according to the invention are excellent for hydrophobizing cementitious construction materials and/or for suppressing efflorescence on surfaces of hardened cementitious construction materials. In this case, the carbonic acid derivatives are added to the non-hardened cementitious construction materials in a quantity of 0.01 to 5 wt % related to the cementitious portion. All concrete and mortar systems having cement as a main bonding agent and optionally lime, hard plaster or anhydrite as side component are to be regarded as the cementitious construction materials according to the invention.

The carbonic acid derivatives according to the invention are directly added to the tempered cementitious construction materials. However, it is also possible within the framework of the present invention that the additives used according to the invention are added to the mixing water or residual water in emulsified form by the aid of external emulsifiers (e.g. ethoxylated compounds, such as fatty acid ethoxylate, ethoxylated caster oil or ethoxylated fatty amine).

The carbonic acid derivatives proposed according to the invention are excellently suitable as means for preventing or suppressing efflorescences on surfaces of hardened cementitious construction materials and/or for hydrophobizing the corresponding cementitious systems.

Furthermore, said cementitious products absorb in hardened state considerably smaller amounts of water as a result of the inventive additives such that frost damage and premature rusting of the reinforcing steel can be significantly reduced.

The following examples shall now explain the invention in detail.

### EXAMPLES

### Example 1

Charge 100 g (0.050 mol) polypropylene glycol 2000 (company Aldrich) at room temperature into the reaction vessel, add 11.03 g (0, 1125 mol) maleic acid anhydride (company Aldrich) and finally add 0.16 g (0.002 mol) sodium acetate (company Aldrich). The reaction chamber is flushed with nitrogen and the reaction mixture is heated to 80° C. This temperature is kept for 5 hours. Subsequently, a cooling process takes place. A slightly orange-colored, slightly viscous liquid is obtained.

### Example 1A

100 g of the reaction product of example 1 are emulsified in 300 g water and are neutralized by a 10% NaOH solution.

A clear slightly viscous 25% aqueous solution is obtained.

### Example 2

Charge 175 g (0.203 mol) of an aminoalkylpolydimethylsiloxane (trade name: Tegomer A-Si 2122, company Tego) at room temperature into a reaction vessel, add 43.1 g (0.44 mol) maleic acid anhydride (company Aldrich) and subsequently add 0.67 g (0.008 mol) sodium acetate (company Aldrich). The reaction chamber is flushed with nitrogen and the reaction mixture is heated to 80° C. This temperature is kept for 5 hours. Subsequently, a cooling process takes place. A slightly brown-colored, slightly viscous liquid is obtained.

### Example 2A

100 g of the reaction product of example 2 are emulsified in 400 g water and are neutralized by a 10% NaOH solution.

A clear brown slightly viscous 20% aqueous solution is obtained.

### Example 3

Charge 320 g (0.517 mol) of a hydroxy polyester (trade name: Sovermol 818, company Cognis) at room temperature into the reaction vessel. Add 142.48 g (1.453 mol) succinic acid anhydride (company Aldrich) and subsequently add 1.7 g (0.021 mol) sodium acetate (company Aldrich). The reaction chamber is flushed with nitrogen and the reaction mixture is heated to 80° C. This temperature is kept for 5 hours. Subsequently, a cooling process takes place. A slightly brown-colored, viscous liquid is obtained.

### Example 3A

100 g of the reaction product of example 3 are emulsified in 400 g water and are neutralized by 10% NaOH solution.

Clear brownish slightly viscous 20% aqueous solution is obtained.

### Example 4

Charge 315 g (0.309 mol) caster oil (company Hanf & Nelles) at room temperature to the reaction vessel, add 98.12 g (1.001 mol) maleic acid anhydride (company Aldrich) and subsequently add 1.01 g (0.012) sodium acetate (company Aldrich). The reaction chamber is flushed with nitrogen and the reaction mixture is heated to 80° C. This temperature is kept for 5 hours. Subsequently, a cooling process takes place. A brown-colored, viscous liquid is obtained.

### Example 5

Charge 280 g (0.156 mol) of an ethoxylated caster oil (trade name Tegotens R20, company Tego) at room temperature into a reaction vessel, add 42.01 g (0.428 mol) maleic acid anhydride (company Aldrich) and subsequently add 0.51 g (0.006 mol) sodium acetate (company Aldrich). The reaction chamber is flushed with nitrogen and the reaction mixture is heated to 80° C. This temperature is kept for 5 hours. Subsequently, a cooling process takes place. An orange-colored, viscous liquid is obtained.

### Example 5A

100 g of the reaction product are emulsified in 400 g water and are neutralized by 10% NaOH solution.

A clear orange-colored slightly viscous 20% aqueous solution is obtained.

### Example 6

Charge 143.45 g (0.547 mol) tall oil fatty acid (company Hanf & Nelles) at room temperature into the reaction vessel. Add 96.3 g (0.264 mol) bisphenol A diglycidylether (trade name: Araldit GY 240; company Huntsman).

Subsequently add 0.25 g (0.78 mol) tetrabutylammoniumbromide (company Aldrich). The reaction chamber is flushed with nitrogen and the reaction mixture is heated to 150° C. This temperature is kept for 8 hours until an acid number of <2 is achieved.

Subsequently, it is cooled down to room temperature and 57.49 g (0.574 mol) succinic acid anhydride (company Aldrich) as well as 0.87 g (0.010 mol) sodium acetate (company Aldrich) is added. The reaction chamber is flushed with nitrogen and the reaction mixture is heated to 80° C. This temperature is kept for 5 hours. Subsequently, a cooling process takes place. A dark red, very viscous liquid is obtained.

### Example 6A

100 g of the reaction product of example 6 are emulsified in 400 g water and with a 10% KOH solution.

A clear red slightly viscous 20% aqueous solution is obtained.

### Example 7

Charge 280 g (0.112 mol) of a hydroxyalkylpolydimethylsiloxane (trade name: Tegomer A-Si 2311, company Tego) at room temperature into the reaction vessel. 23.78 g (0.243 mol) Add maleic acid (company Aldrich) and subsequently add 0.25 g (0.003 mol) sodium acetate (company Aldrich). The reaction chamber is flushed with nitrogen and the reaction mixture is heated to 80° C. This temperature is kept for 5 hours. Subsequently, a cooling process takes place. A slightly brown-colored slightly viscous liquid is obtained.

### Example 7A

100 g of the reaction product of example 7 are emulsified in 400 g water and are neutralized by a 10% KOH solution.

A milky brownish slightly viscous 20% aqueous solution is obtained.

### Example 8

Charge 300 g (0.150 mol) of a polyoxypropyleneamine (trade name: Jeffamine D-2000, company Huntsman) at room temperature into the reaction vessel. Add 32.42 g (0.324 mol) succinic acid anhydride (company Aldrich) and subsequently add 0.33 g (0.004 mol) sodium acetate (company Aldrich). The reaction chamber is flushed with nitrogen and the reaction mixture is heated to 80° C. This temperature is kept for 5 hours. Subsequently, a cooling process takes place. A brown-colored viscous liquid is obtained.

### Example 8A

100 g of the reaction product of example 8 are emulsified in 400 g water and are neutralized by a 10% NaOH solution.

A clear brownish slightly viscous 20% aqueous solution is obtained.

### Example 9

Charge 360 g (0.05 mol) ethylene diamine tetrakis(polyethylene glycol-b-polypropylene glycol)-tetrol (company Aldrich) at room temperature into the reaction vessel. Add 24.66 g (0.22 mol) itacon acid anyhdride (company Aldrich) and subsequently add 0.25 g (0.003 mol) sodium acetate (company Aldrich). The reaction chamber is flushed with nitrogen and the reaction mixture is heated to 80° C. This temperature is kept for 5 hours. Subsequently, a cooling process takes place. A light brown viscous liquid is obtained.

### Example 9A

100 g of the reaction product of example 9 are emulsified with 400 g water and are neutralized by a 10% NaOH solution.

A clear brownish slightly viscous 20% aqueous solution is obtained.

### Examination of the Products Manufactured

The specimens are manufactured according to the following method and are examined with respect to their efflorescence behavior.

According to standard, a mixture (11 kg) is manufactured according to the following recipe in a compulsory mixer, wherein first of all, all aggregates are mixed dry for 10 seconds. Subsequently, the base water is added and mixed for 2 minutes. Then the residual water is added. Mixing period: 2 minutes. The additive is added to the residual water:

| | | |
|---|---|---|
| 380 | kg/m³ | cement (Bernburg CEM I 42.5 R; 380 kg/m³) |
| 1104 | kg/m³ | sand 0/2 |
| 296 | kg/m³ | gravel 2/5 |
| 296 | kg/m³ | gravel 5/8 |
| 137 | kg/m³ | water |
| w/z: | | 0.36 |

The additive is used in different dosages related to the cement in the mixture and is either added to the residual water or to the concrete mixture. The statements concerning the dosage of the additive always refer to the solid “additive” to solid “cement”. The water content of the additive is subtracted from the quantity of the batching water.

To manufacture the specimens, exactly 1300 g of the green concrete mixture are supplied into round moulds and are compressed by a bearing weight of 30 kg on a vibrating table for a period of 90 seconds. Subsequently, the green specimen is removed from the mould and stored in a climatic chamber (20°, 65% relative air moisture) for hardening for 2 days. Subsequently, the luminance of the specimen is measured (L1) by a color photo spectrometer (Color-Guide sphere spin, Byk Gardner), wherein a template with 9 measuring points is laid onto the specimens, so that later during the 2^{nd} measurement the same points are measured. The mean value L1 results from these 9 points. Subsequently, the stones are dipped into distilled water for a period of approx. 2 seconds and are packed in an airtight manner in moist state into a plastic bag. This bag is stored in the climatic chamber for 10 days. Subsequently, the stones are unpacked and stored for drying in the climatic chamber for 2 days. Now the luminance of the specimens is measured a second time by the template and color photo spectrometer (L2). 6 specimen are produced per mixture (and the mean value is formed therefrom). The color change of the surface (ΔL) of the specimens (increase of the degree of white) results as: ΔL=L2−L1

Besides the lucency (ΔL) of the specimen caused by the efflorescence, the homogeneity of the surface was also evaluated and the water absorption of the specimen was determined.

Determination of the water absorption (WA) based on EN ISO 15148:

The dry and hardened specimen are weighed (W1) and put into a water quench in a manner that the lower side rests on the point supports and does not contact the container bottom. The water level is approx. 5 mm above the highest portion of the lower side. After 15 minutes the specimens are taken out of the water bath and are weighed a second time (W2). The specimen is previously dried with a moist wrung out sponge. The water absorption results as: WA=W2−W1.

TABLE 1
(Accelerated efflorescence in the climatic chamber, 20° C., 65% rel. air
humidity)

| | | Luminance | | | | | | | |
|---|---|---|---|---|---|---|---|---|---|
| | Dosage | difference | | | Water absorption | | | Analysis of | |
| Examp. | [wt %] | ΔL | | | WA [g] | | | the surface | addition |
| 1 | 0.40 | 0.9 | (7.6) | −88% | 3.5 | (46.7) | −91% | faultless | BM |
| | 0.20 | 1.2 | (7.6) | −84% | 3.5 | (46.7) | −90% | faultless | BM |
| 1A | 0.40 | 0.8 | (7.6) | −89% | 3.5 | (46.7) | −92% | faultless | RW |
| | 0.20 | 1.1 | (7.6) | −86% | 3.5 | (46.7) | −90% | faultless | RW |
| 2 | 0.40 | 1.0 | (8.8) | −89% | 4.4 | (52.5) | −92% | faultless | BM |
| | 0.20 | 1.2 | (8.8) | −86% | 5.0 | (52.5) | −90% | faultless | BM |
| 2A | 0.40 | 0.9 | (8.8) | −90% | 4.3 | (52.5) | −92% | faultless | RW |
| | 0.20 | 1.1 | (8.8) | −88% | 4.7 | (52.5) | −91% | faultless | RW |
| 3 | 0.40 | 0.8 | (9.1) | −91% | 3.9 | (48.9) | −92% | faultless | BM |
| | 0.20 | 1.1 | (9.1) | −88% | 4.5 | (48.9) | −91% | faultless | BM |
| 3A | 0.40 | 0.7 | (9.1) | −92% | 3.9 | (48.9) | −92% | faultless | RW |
| | 0.20 | 1.0 | (9.1) | −89% | 4.8 | (48.9) | −90% | faultless | RW |
| 4 | 0.40 | 1.1 | (8.3) | −87% | 4.3 | (57.3) | −92% | faultless | BM |
| | 0.20 | 1.4 | (8.3) | −83% | 5.0 | (57.3) | −91% | faultless | BM |
| 5 | 0.40 | 0.9 | (7.9) | −89% | 3.8 | (53.9) | −93% | faultless | BM |
| | 0.20 | 1.3 | (7.9) | −84% | 4.8 | (53.9) | −91% | faultless | BM |
| 5A | 0.40 | 0.8 | (7.9) | −90% | 3.6 | (53.9) | −93% | faultless | RW |
| | 0.20 | 1.1 | (7.9) | −86% | 4.4 | (53.9) | −92% | faultless | RW |
| 6 | 0.40 | 1.1 | (8.5) | −87% | 4.2 | (56.1) | −93% | faultless | BM |
| | 0.20 | 1.3 | (8.5) | −85% | 5.0 | (56.1) | −91% | faultless | BM |
| 6A | 0.40 | 0.9 | (8.5) | −89% | 4.3 | (56.1) | −92% | faultless | RW |
| | 0.20 | 1.1 | (8.5) | −87% | 5.2 | (56.1) | −91% | faultless | RW |
| 7 | 0.40 | 1.0 | (8.9) | −89% | 3.6 | (54.0) | −93% | faultless | BM |
| | 0.20 | 1.3 | (8.9) | −85% | 4.3 | (54.0) | −92% | faultless | BM |
| 7A | 0.40 | 0.9 | (8.9) | −90% | 3.9 | (54.0) | −93% | faultless | RW |
| | 0.20 | 1.2 | (8.9) | −87% | 4.7 | (54.0) | −91% | faultless | RW |
| 8 | 0.40 | 1.1 | (8.1) | −86% | 3.4 | (50.5) | −93% | faultless | BM |
| | 0.20 | 1.3 | (8.1) | −84% | 4.0 | (50.5) | −92% | faultless | BM |
| 8A | 0.40 | 0.8 | (8.1) | −90% | 3.7 | (50.5) | −93% | faultless | RW |
| | 0.20 | 1.2 | (8.1) | −85% | 4.4 | (50.5) | −91% | faultless | RW |
| 9 | 0.40 | 1.3 | (8.4) | −85% | 3.8 | (49.2) | −92% | faultless | BM |
| | 0.20 | 1.5 | (8.4) | −82% | 4.3 | (49.2) | −91% | faultless | BM |
| 9A | 0.40 | 1.1 | (8.4) | −87% | 3.5 | (49.2) | −93% | faultless | RW |
| | 0.20 | 1.4 | (8.4) | −83% | 4.2 | (49.2) | −91% | faultless | RW |

BM: addition of the additive to the concrete mixture
RW: addition of the additive to the residual water
The values in brackets are the results of the null mixtures (without additive).
The percentage values define about how much the additive has reduced the luminance or water absorption compared to null mixtures (without additive).
The dosages define the solid of the additive related to the cement in the mixture.

1. Use of a carbonic acid derivative, which can be obtained by conversion of an unsaturated dicarbonic acid anhydride (A) with a hydrophobic reactive component (B), which has at least one group reactive with respect to a carbonic acid anhydride and which has a mean molecular weight of 200 to 50,000 Dalton, selected from the group of
(B)(i) a polydimethyl siloxane of the general formula (I) 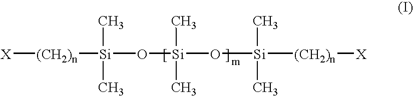
wherein
X=OH, NH₂, SH, NHR¹,
R¹=H, CH₃, C₂H₅,
m=1 to 50 and
n=1 to 6
or
(B)(ii) a polypropylene glycol of the general formulae (IIa) and/or (IIb) and/or (IIc) 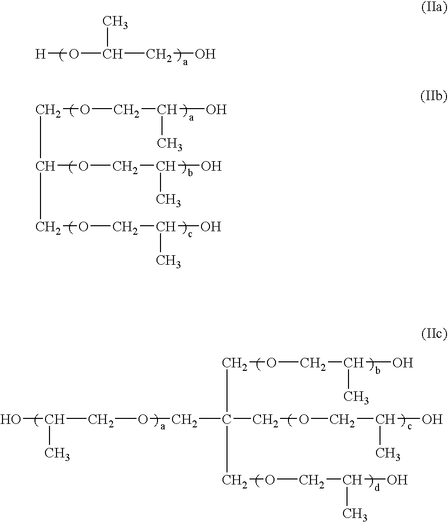
wherein
a, b, c and d independent of one another mean 1 to 150
or
(B)(iii) a polyoxyalkylene anine of the general formulae (IIIa) and/or (IIIb) and/or (IIIc) 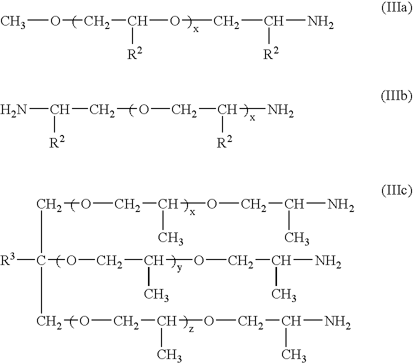
wherein
R²=H, CH₃,
R³H, CH₃, C₂H₅, and
x, y and z independent of one another mean 1 to 100
or
(B)(iv) a polyalkylene glycol based on alkylene diamines of the general formula (IV) 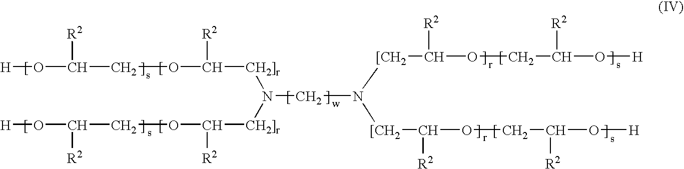
wherein
w=2 to 12,
r and s represent independent of one another 1 to 150 and
R² has the above-mentioned meaning
or
(B)(v) a triglyceride based on at least one hydroxy fatty acid, wherein the hydroxy fatty acid can be etherized
or
(B)(vi) an epoxy derivative produced by conversion of a di-, tri- or tetraglycidyl component (C)(i) with a possibly unsaturated reactive component (C)(ii), composed of a C₈-C₂₈-fatty acid, a C₈-C₂₈-Alcohol or a C₈-C₂₈-secondary amine for rendering cementitious products hydrophobic.

2. Use as claimed in claim 1, characterized in that maleic acid anhydride or itacon acid anhydride is used as unsaturated dicarbonic acid anhydride.

3. Use as claimed in claim 1, characterized in that the reactive component (B) has an average molecular weight of 500 to 10,000 Dalton.

4. Use as claimed in claim 1, characterized in that m in formula (I) is 10 to 30.

5. Use as claimed in claim 1, characterized in that the reactive component (B)(v) is consisting of a triglyceride derivative on the basis of hydroxy fatty acids, selected from the group of ricinoleic acid, cerebronic acid, nemotin acid or 12-hydroxy stearic acid.

6. Use as claimed in claim 1, characterized in that glyceride compounds selected from the group of cyclohexane dimethanol diglycidylether, glycerine triglycidylether, neopentylglycol diglycidylether, pentaerythritol tetraglycidylether, 1,6-hexanediol diglycidylether, polypropylene glycol diglycidylether, polyethylene glycol diglycidylether, trimethylolpropane triglycidylether, bisphenol A diglycidylether, bisphenol F diglycidylether, 4,4-methylenebis (N,N-diglycidylaniline), tetraphenylolethane glycidylether, N,N-diglycidylaniline, diethyleneglycol diglycidylether, 1,4-butanediol diglycidylether or mixtures thereof are used as di-, tri- or tetraglycidyl compound (C)(i).

7. Use as claimed in claim 1, characterized in that fatty acids from the group of tall oil fatty acid, stearic acid, palmitic acid, sunflower oil fatty acid, coconut oil fatty acid (C₈-C₁₈), coconut oil fatty acid (C₁₂-C₁₈), soy bean oil fatty acid, linseed oil fatty acid, dodecane acid, oleic acid, linoleic acid, palm kernel oil fatty acid, palm oil fatty acid, linolenic acid and/or arachidonic acid are used as reactive component (C)(ii).

8. Use as claimed in claim 1, characterized in that alkanoles from the groups of 1-eicosanol, 1-octadecanol, 1-hexadecanol, 1-tetradecanol, 1-dodecanol, 1-decanol, 1-octanol are used as reactive component (C)(ii).

9. Use as claimed in claim 1, characterized in that alkyl amines from the group of 2-ethylhexylamine, dipentylamine, dihexylamine, dioctylamine, bis(2-ethylhexyl)amine, N-methyloctadecylamine, didecylamine are used as reactive component (C)(ii).

10. Use as claimed in claim 1, characterized in that the molar ratio of the unsaturated dicarbonic acid anhydride (A) with respect to the reactive component (B) is 0.1 to 1.0 mol (A) per mol of the reactive group of component (B).

11. Use as claimed in claim 1, that the carbonic acid derivatives are used for suppressing efflorescences of hardened cementitious products.

12. Use as claimed in claim 1, characterized in that the carbonic acid derivatives of the non-hardened cementitious products are added in a quantity of 0.01 to 5 wt % related to the cementitious portion.

13. Carbonic acid derivatives, obtained by reacting an unsaturated dicarbonic acid anhydride (A) with a hydrophobic reactive component (B), which has at least one group reactive with respect to carbonic acid anhydrides and which has a mean molecular weight of 200 to 50,000 Dalton, selected from the group of
(B)(ii) a polypropylene glycol of the general formulae (IIb) and/or (IIc) 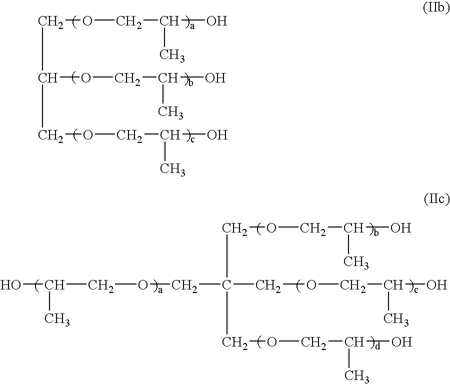
wherein
a, b, c and d mean independent of one another 1 to 150
or
(B)(iii) a polyoxyalkylene amine of the general formulae (IIIa) and/or (IIIc) 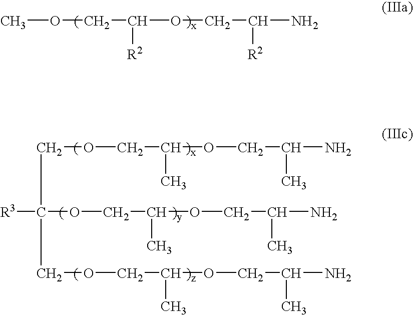
wherein
R²=H, CH₃,
R³=H, CH₃, C₂H₅, and
x, y and z mean independent of one another 1 to 100
or
(B)(v) a triglyceride based on at least a hydroxy fatty acid, wherein the hydroxy fatty acid can be etherified by 1 to 100 mol of an ethylene oxide derivative
or
(B)(vi) an epoxy derivative, which was produced by reacting a di-, tri- or tetraglycidyl component (C)(i) with an optionally unsaturated reactive component (C)(ii) consisting of a C₈-C₂₈-fatty acid, a C₈-C₂₈-alcohol or a C₈-C₂₈-secondary amine.

14. Carbonic acid derivatives as claimed in claim 13, characterized in that maleic acid anhydride, succinic acid anhydride or itacon acid anhydride is used as an unsaturated dicarbonic acid anhydride.

15. Carbonic acid derivatives as claimed in claim 13, characterized in that the reactive component (B) has an average molecular weight of 500 to 10,000 Dalton.

16. Carbonic acid derivatives as claimed in claim 13, characterized in that the reactive component (B)(v) is consisting of a triglyceride derivative on the basis of hydroxy fatty acids selected from the group of ricinoleic acid, cerebronic acid, nemotin acid or 12-hydroxy stearic acid.

17. Carbonic acid derivatives as claimed in claim 13, characterized in that glycidyl compounds, selected from the group of cyclohexane dimethanol diglycidyl ether, glycerine triglycidyl ether, neopentylglycol diglycidylether, pentaerythritol tetraglycidylether, 1,6-hexanediol diglycidylether, polypropylene glycol diglycidylether, polyethylene glycol diglycidylether, trimethylol propane triglycidylether, bisphenol A diglycidylether, bisphenol F diglycidylether, 4,4-methylenebis (N,N-diglycidylaniline), tetraphenylolethane glycidylether, N,N-diglycidylanilinc, diethyleneglycol diglycidylether, 1,4-butanediol diglycidylether or mixtures thereof are used as di-, tri- or tetraglycidyl compound (C)(i).

18. Carbonic acid derivatives as claimed in claim 13, characterized in that fatty acids of the group of tall oil fatty acid, stearic acid, palmitic acid, sunflower oil fatty acid, coconut oil fatty acid (C₈-C₁₈), coconut oil fatty acid (C₂-C₁₈), soy bean oil fatty acid, linseed oil fatty acid, dodecane acid, oleic acid, linoleic acid, palm kernel oil fatty acid, palm oil fatty acid, linolenic acid and/or arachidonic acid are used as reactive component (C)(ii).

19. Carbonic acid derivative as claimed in claim 13, characterized in that alkanoles from the group of 1-eicosanol, 1-octadecanol, 1-hexadecanol, 1-tetradecanol, 1-dodecanol, 1-decanol, 1-octanol are used as reactive component (C)(ii).

20. Carbonic acid derivatives as claimed in claim 13, characterized in that alkylamines of the group 2-ethylhexylamine, dipentylamine, dihexylamine, dioctylamine, bis(2-ethylhexyl)amine, N-methyloctadecylamine, didecylamine are used as reactive component (C)(ii).

21. Carbonic acid derivates as claimed in claim 13, characterized in that the molar ratio of the unsaturated dicarbonic acid anhydride (A) to the reactive component (B) is 0.1. to 1.0 mol per mol of the reactive group of component (B).

22. Method of manufacturing the carbonic acid derivatives as claimed in claim 13, characterized in that the unsaturated dicarbonic acid derivative (A) with the reactive component (B) is converted without the use of a solvent in a temperature range of 20 to 150° C. optionally in the presence of a catalyst.

23. Method as claimed in claim 22, characterized in that an alkaline or alkaline earth salt of an organic acid such as sodium acetate or potassium acetate is used as a catalyst.
